(19) United States
(12) Patent Application Publication
(10) Pub. No.: US 2005/0036966 A1
(43) Pub. Date: Feb. 17, 2005

(54) Medicine for preventing and treating bromidrosis

(76) Inventor: Marc Heckmann, Pocking (DE)

Correspondence Address: Steven L Highlander;Fulbright & Jaworski, 600 Congress Avenue, Suite 2400, Austin, TX, 78701, US

(21) Appl. No.: 10/490,244

(22) PCT Filed: Sep. 23, 2002
(86) PCT No.: PCT/DE02/03561

(30) Foreign Application Priority Data
Sep. 21, 2001 (DE) 10146647.1

(51) Int. Cl. A01N 25/00; A61K 7/32

(52) U.S. Cl. 424/65

(57) ABSTRACT

The present invention relates to the use of botulinus toxin for the manufacture of a medicament for prophylaxis and/or therapy of bromhidrosis as well as for manufacture of a cosmetic means for the improvement of the body odour.

The present invention relates to the use of botulinus toxin for the manufacture of a medicament for prophylaxis and/or therapy of bromhidrosis as well as for manufacture of a cosmetic means for the improvement of the body odour.

In cosmetics there are methods for generating a pleasant odour with foreign substances (perfumes); in medicine there are methods for the reduction of a pathological malodorant body odour (bromhidrosis). However, either of them is not distinct from the possibility to increase the quality rating or acceptability of the body's odour.

Body odour is a generally known and widespread phenomenon. It can occur in different individuals under comparable conditions in very different intensities and be perceived differently by the affected people themselves as well as by their fellow men.

In contrary to the common idea the body sweat, i.e. the secretion of the eccrine perspiratory glands, is totally odourless. Therefore excessive perspiration must not be confused with excessive body odour. In its composition sweat is a clear aqueous fluid consisting predominantly of sodium-, potassium-, calcium-, magnesium- and chloride ions and besides that contains lactate, urea and traces of amino acids, biogenic amines and vitamins. Under exceptional circumstances medicaments can be excreted via the sweat such as griseofulvin and ketokonazol but which does not play a significant role for the body odour. The more sweat is produced, for example under extreme conditions like in the sauna, the more the sweat is diluted, i.e. all the more aqueous the sweat becomes. An excessive unnatural production of sweat under normal physiological conditions is known as a disease pattern, the so-called hyperhidrosis. Therefore the excessive sweating (hyperhidrosis) is not the direct cause for body odour. On the contrary: it was explicitly emphasised in medical textbooks and publications that patients suffering from excessive underarm perspiration (Hyperhidrosis axillaris) typically do not develop a strong body odour [4]. This is explained by the fact that the odourless, in huge quantities outpouring sweat quasi “flushes away” the odour producing substances from the skin. However, in contrast to the eccrine perspiratory glands the so-called scent glands (apocrine and apo-eccrine glands) can release a discernible smelling secretion. This secretion can in turn be converted into malodorant substances on the surface of the skin by bacteria. The scent glands can therefore be assigned a causative role in the occurrence of the body odour. Scent glands can be found in great numbers e.g. in the human armpit, but they get active usually only after puberty. Accordingly, the body odour is usually much stronger in adults than in children.

In a simplified way body odour can be caused by two essential factors:
1. The secretions produced by the scent glands show a characteristic odour being perceived by other people in very different extents.
2. The secretions of the scent glands, derivatives of steroids and other body substances like e.g. the grease of the skin, can be degraded by the microflora of the skin resulting in a series of products of decomposition. Such products of decomposition generated by bacteria can for example produce a penetrative or rancid odour. Certain amino acids can account for the body odour, too.

Thus the body odour is in summary mostly a matter of the mixture of different components which are not yet totally analysed in their composition and which individually can be very different. For this reason, analyses concerned with the body odour, are not performed only by biochemical measurements but independent test persons are adopted who perform an assessment of the body odour by their olfactory organ and by this means indicate on scales how intensive or how unpleasant a certain odour is.

In the case of an extremely strong and unpleasant occurrence, the body odour is defined as a disease named with the term “bromhidrosis”. However, body odour can also be perceived in a wide range of perceptions between unpleasant, disgusting up to pleasant or even infatuating and stimulating. Thus, in behavioural psychology the effect of body odour is not judged only by the intensity (how strong does something smell) but also the emotional valence (how good or how bad does something smell).

The measures known by now for obtaining a pleasant body odour consist basically in the use of perfumes, fragrants, substances and deodorants for covering the own body odour. Moreover therapies for the reduction of an unpleasant body odour are primarily aimed against bacteria of the skin and their activity in decomposition on the skin. Thus, in the case of bromhidrosis are recommended for example:
frequent washing and changing of the underwear
frequent application of soaps or syndets for rinsing of intensively smelling substances
application of deodorants
disinfectants or skin cleansers that reduce and destroy, respectively, the bacterial flora of the skin

The disadvantage of the above mentioned measures is: the more intensively they are applied, the more they lead to an irritation of the skin and to a disturbance of the skin's function as a barrier which may lead to unpleasant eczematoid reactions accompanied by redness and itching. In addition specially the fragrant substances in deodorants exhibit a high allergenic power abetting an immunological allergisation which may lead to livelong existing allergies.

Although the suppression of the secretion of sweet e.g. by the use of metal-salt containing solutions such as aluminium chloride, is recommended in order to bring about a mechanical obstruction of the channels of the perspiratory glands thereby repressing the sweat flow. However, these and similar therapies lead also often to unwanted irritations of the skin. Furthermore they are not suited to affect the body odour directly taking the above mentioned odourless features of the sweat into account, but may at best accomplish an indirect change of the dermal environment.

Thus, in summary all conventional measures disregard the importance of the scent glands and their manipulation for the improvement of the body odour.

Therefore the problem of the invention consists in the provision of a medicament for the treatment of bromhidrosis and in the provision of a cosmetic means for the improvement of the body odour.

The problem is solved by the subject-matter defined in the patent claims.

The invention is illustrated by the following figures.

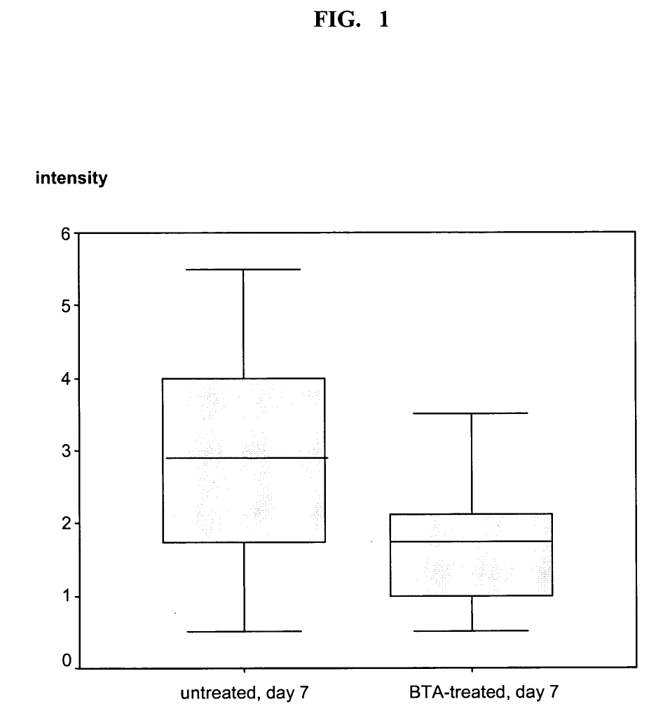

FIG. 1 shows the assessment of the intensity of the body odour (0=no odour perceptible, 6=maximally intensive odour) after one-sided treatment with botulinus toxin in a diagram. Control-treated armpits: n=16, median=2.88, SD=1.43. Botulinus toxin-A-treated armpits: n=16, median=1.75, SD=0.86. Significance of the difference (Wilcoxon-test): p=0.02. BTA means botulinus toxin-A.

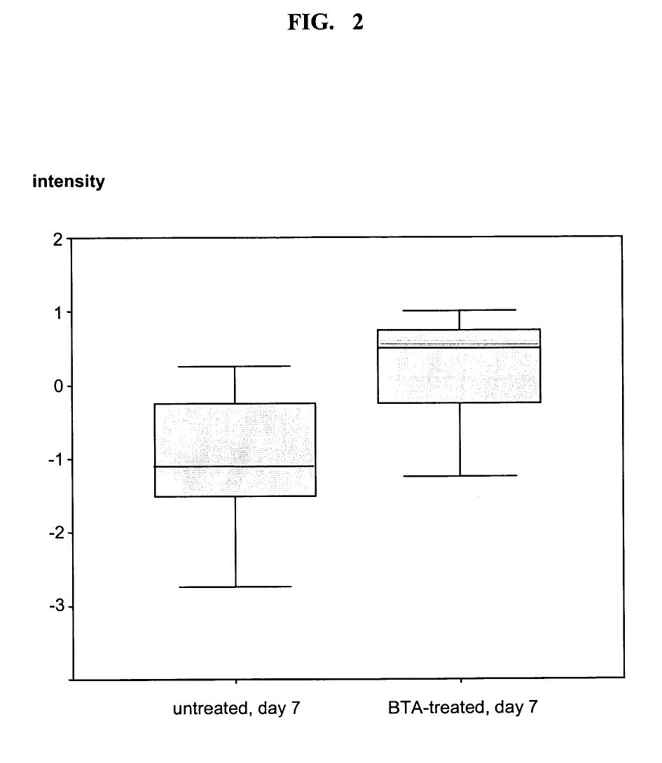

FIG. 2 shows the assessment of the quality rating (valency) of the body odour (−3=extremely unpleasant; +3=extremely pleasant) after one-sided treatment with botulinus toxin in a diagram. Control-treated armpits: n=16, median=−1.13, SD=0.89. Botulinus toxin-A-treated armpits: n=16, median=0.5, SD=0.75. Significance of the difference (Wilcoxon-test): p=0.001. BTA means botulinus toxin-A.

One aspect of the present invention relates to the use of botulinus toxin for the manufacture of a medicament for prophylaxis or therapy of bromhidrosis. A further aspect relates to the use of botulinus toxin for the preparation of a cosmetic agent for the improvement of the body odour.

Therefore the present invention relates to a new method for the manipulation of the body odour e.g. in terms of a reduction (less intensive) and improvement (sensed as more pleasant) of the body odour. Thereby it is not about a mere reduction of the secretion of sweat since sweat is an odourless fluid but it is about a change in the features of the smell of the armpit or other areas of the skin which generate a perceptible and unpleasant odour.

Botulinus toxins are a group of highly potent bacterial toxins being produced by Clostridium botulinum under unaerobic conditions. The subtype botulinum toxin-A is approved as a medicamentous agent for the treatment of selected neuromuscular diseases in the US since 1989 and in Germany since 1991 and 1993, respectively. In Germany botulinus toxin-A is available under the trade name Botox® (distribution by the company Merz, Frankfurt; manufacture: Allergan, Irvine Calif., USA) and under the trade name Dysport® (distribution by the company Ispen-pharma, Ettlingen). Since 2001 a further preparation named NeuroBloc® (company Elan, Munich) has been available which contains the subtype botulinus toxin-B.

The pharmacology, pharmaceutical manufacture as well as numerous clinical applications of botulinus toxin are elaborately described in the technical literature [1, 2]. The clinical effect of the botulinus toxins is due to a blockade of the release of acetylcholine. Therefore all nerve endings can be blocked which use acetylcholine as a transmitter.

The successful application of botulinus toxin-A in treatment of the excessive sweating (hyperhidrosis) has been repeatedly described in the technical literature [3]. On the contrary, the influence on the body odour was not known up to now. In fact, it was discussed in a scientific publication on the treatment of hyperhidrosis that the injection of botulinus toxin had no influence on the body odour [3].

However, it was found surprisingly by the inventor in clinical observations that botulinus toxin is effective in the case of bromhidrosis and can even improve the body odour of healthy people. The latter is by no means the corollary of a successful bromhidrosis therapy since the reduction of an unpleasant or even pathological body odour should merely lead to a less disturbing or at best neutral body odour but not to the generation of an independent positive body odour.

Therefore one aspect of the present invention consist in the provision of a substance leading to the fact that the body's own odour displays an increasingly positive and more pleasant effect, respectively, on other fellow men and therefore gives a competitive edge to the user in the case of all interpersonal relations in which the olfactory perception plays a direct or an indirect role.

In this case the botulinus toxin can not only be used in its wild type form but also its derivatives, fragments or a botulinus toxin with sundry changes e.g. chemical modifications. Thereby “derivatives” means that the amino acid sequence of the botulinus toxin may contain substitutions, deletions, insertions or additions. Thereby “fragments” means that only certain parts of the botulinus toxin may be used as long as these parts display the biological activity of the wild type botulinus toxin.

Preferably the botulinus toxin is introduced into the skin via intracutaneous injection. This can be accomplished for example by the use of a syringe with acute hypodermic needles and gauge needles (e.g. 30 gauge), respectively, or by any other method for injection (e.g. high pressure and needle less injection, respectively). The injections are evenly distributed e.g. in a distance of 0.5 to 5 cm over the area of skin to be treated. Other injections e.g. subcutaneous or intra epidermal ones are possible as well.

Also other forms of application such as spreading of the botulinus toxin in a suitable preparation (e.g. gel, creme, ointment, spray) with or without additives assisting the penetration of skin, are suited, as long as a transcutaneous absorption of the active agent is provided. Equally, the active agent can be applied on and introduced into the skin, respectively, using a water bath or a bath of an ulterior solvent with or without the application of feeble current (iontophoresis).

Preferably a ready-for-use injection solution of botulinus toxin is prepared. Such can be prepared e.g. by dissolving of one packaging unit of the preparation Botox® or of the preparation Dysport® in sterile physiological saline solution (e.g. 1-10 ml or a volume freely to be determined). Or the preparation Neurobloc® is used which is available already in a dissolved form. Or any subtypes of botulinus toxins (e.g. A, B, C, D, E, F, G) and derivatives, fragments or forms of these botulinus toxins are used, respectively, which are changed in any respect. Or combinations of several botulinus toxin subtypes or combinations with other substances and auxiliary substances, respectively, are used which are suited for one of the above mentioned modes of application. The concentration of the active agent in the solution (determined in mouse units per ml) can be chosen freely according to the individual needs and experiences.

Preferably, the armpit is suited for the treatment, however, any other region of the body can be treated with it such as the inguinal region, the gluteal region, the feet. Any form of a supporting pre- or after-treatment, e.g. by spreading of an analgetic creme, cooling, maceration of the skin or a creme or ointment changing the odour, or other external applications of any kind can be combined with one of the above mentioned forms of application.

The following examples serve for illustration and are not to be mistaken as limiting the invention.

### EXAMPLE 1

### Casuistics

A patient with strong body odour emanating from the armpit received 50 units Botox® solved in 2 ml NaCl distributed on 10 intracutaneous injection points per armpit. After one week he detected a considerable decrease of the intensity of his body odour though having the same hygienic habits.

### EXAMPLE 2

### Pilot Study

A group of 16 test persons was examined after education and acquiescence in written form. Each test person was asked not to use a deodorant, perfume or perfume soap etc., not to eat onions, asparagus or garlic and not to have intimate or close contact with partners for three days. On the third day each test person was asked to wear a white T-shirt (100% cotton, pre-washed) for 24 hours from noon time to noon time. After this the underarm parts of the T-shirts were cut out and put separately into air-tightly lockable glass flasks. These were labelled anonymously and then presented to the participants as olfactory samples with each participant taking a smell of all olfactory samples without knowing whom they were derived from. After this each participant received an injection treatment in both armpits with ten cut-in points per side. On the one side thereby 100 units botulinus toxin-A (Dysport®) solved in 2 ml isotonic NaCl-solution where administered and on the other side 2 ml isotonic saline solution. Neither the physician nor the participant knew which side was treated with the active agent or with the control solution (double-blind). After one week the T-shirt olfactory test was repeated under exactly the same conditions. After the statistical analysis a highly significant difference between the both sides was to be found: the armpits treated with botulinus toxin and the T-shirt cut-outs, respectively, smelled less intensive and less unpleasant and more pleasant, respectively, than the controls.

### EXAMPLE 3

### Pilot Study

56 women were asked to assess the olfactory samples of 16 foreign donors. The methodology for the preparation of the olfactory samples was analogous to example 2, i.e. there were two samples from each donor: one from the botulinus toxin-treated armpit and one from the untreated armpit.

The women were asked to assess the following:
1. Which of the two samples smells more pleasant (double-blind approach)?
2. How is the olfactory quality: (7 point-scale reaching from −3=very unpleasant, over 0=neutral up to +3=very pleasant)?
3. How would you describe the odour (positive or negative adjectives were given for choice, e.g. bloomy versus purulent, fruity versus rancid, etc.)?
4. How do you feel while perceiving this odour (9 points of valency)?
5. Can you imagine having a partner with this odour?
6. What falls into your mind concerning this odour?

Analysis: The pair-wise comparisons were calculated by means of the McNemar χ²-test for repeated measurements of nominal data points.

The odour of the side which was botulinus toxin-treated was high significantly sensed as more pleasant (p<0.001) and this both in direct comparison and also according to the point-scale. Positive adjectives were high significantly used more often for the botulinus toxin-treated samples. The women felt high significantly “securer” and “happier” in the case of the perception of the botulinus toxin-treated samples and they could also high significantly imagine more often to have a partner with the odour of a botulinus toxin-treated armpit than with the odour of an untreated one (each p<0.001).

1. A method for the prophylaxis and/or therapy of bromhidrosis as well as for the improvement of the general body odour comprising administering a botulinus toxin to a subject having bromhidrosis in an amount sufficient to reduce general body odour.

2. A method for the improvement of the general body odour comprising administering a botulinus toxin to a subject have unpleasant body odour in an amount sufficient to improve general body odour.

3. The method according to claim 1, wherein the botulinus toxin is selected from botulinus toxin type A, B, C, D, E, F and G or derivatives or fragments thereof.

4. The method according to claim 2, wherein the botulinus toxin is selected from botulinus toxin type A, B, C, D, E, F and G or derivatives or fragments thereof.

5. The method according to claim 1, wherein the botulinus toxin is administered by injection of skin in the area to be treated.

6. The method according to claim 2, wherein the botulinus toxin is administered by injection of skin in the area to be treated.

7. The method according to claim 1, wherein the botulinus toxin is administered by spreading on the skin in the area to be treated.

8. The method according to claim 2, wherein the botulinus toxin is administered by spreading on the skin in the area to be treated.

9. The method according to claim 1, wherein the botulinus toxin is administered by immersion of the area to be treated in a bath comprising the toxin.

10. The method according to claim 2, wherein the botulinus toxin is administered by immersion of the area to be treated in a bath comprising the toxin.
